(19) United States
(12) Patent Application Publication
(10) Pub. No.: US 2025/0084131 A1
(43) Pub. Date: Mar. 13, 2025

(54) T-CELL EPITOPE POLYPEPTIDE OF FOOT AND MOUTH DISEASE VIRUS AND USES THEREOF

(71) Applicant: Lanzhou Veterinary Research Institute Chinese Academy of Agricultural Sciences, Lanzhou (CN)

(72) Inventors: Shiqi SUN, Lanzhou (CN); Huichen GUO, Lanzhou (CN); Suyu MU, Lanzhou (CN); Shaobin SHANG, Lanzhou (CN); Lingbo CHEN, Lanzhou (CN); Yun ZHANG, Lanzhou (CN); Manyuan BAI, Lanzhou (CN); Jinen WU, Lanzhou (CN); Shuanghui YIN, Lanzhou (CN); Zhidong TENG, Lanzhou (CN); Jingjing ZHOU, Lanzhou (CN); Hu DONG, Lanzhou (CN)

(21) Appl. No.: 18/824,867

(22) Filed: Sep. 4, 2024

(30) Foreign Application Priority Data
Sep. 13, 2023 (CN) 202311180688.1

(51) Int. Cl. C07K 14/005; A61K 39/135

(52) U.S. Cl. CPC C07K 14/005; A61K 39/135; C12N 2770/32122; C12N 2770/32134

(57) ABSTRACT

A T-cell epitope polypeptide of foot and mouth disease virus (FMDV) having an amino acid sequence represented by one of SEQ ID Nos: 1-11: SEQ ID NO: 1: KGKPFNSKVIIATTNLYS; SEQ ID NO: 2: PLQNVYQLVQEVIDRVEL; SEQ ID NO: 3: VVMDDLGQNPDGKDFKYF; SEQ ID NO: 4: YNQQTVVVMDDLGQNPDG; SEQ ID NO: 5: GRTDSVWYCPPDPDHFDG; SEQ ID NO: 6: RGKSGQGKSFLANVLAQA; SEQ ID NO: 7: PDFNRLVSAFEELATGVK; SEQ ID NO: 8: AIRTGLDEAKPWYKLIKL; SEQ ID NO: 9: MSTKHGPDFNRLVSAFEE; SEQ ID NO: 10: MLDGRTMTDSDYRVF; and SEQ ID NO: 11: VLDEVIFSKHKGDTK.

### CROSS-REFERENCE TO RELATED APPLICATIONS

Pursuant to 35 U.S.C.§ 119 and the Paris Convention Treaty, this application claims foreign priority to Chinese Patent Application No: 202311180688.1 filed Sep. 13, 2023, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Aug. 27, 2024, is named LZSY-01001-UUS.xml, and is 10,540 bytes in size.

### BACKGROUND

The disclosure relates to the field of veterinary biologics, more particularly, to a T-cell epitope polypeptide of foot-and-mouth disease virus and its uses.

Foot and Mouth Disease (FMD) is an acute, febrile, high-contact infectious disease of humans and animals caused by the Foot and Mouth Disease Virus (FMDV). FMDV belongs to the family of small RNA viruses without capsular membranes; there are 7 serotypes: A, O, C, SAT1, SAT2, SAT3, and Asial. FMDV inactivated vaccine, as the first animal vaccine successfully developed by human beings and put into use, has made a significant contribution to the prevention and control of the FMD epidemic and the purification of the virus. With the widespread use of the vaccine, the disadvantages of the vaccine have become increasingly apparent, namely, the production of the vaccine requires the use of a live virus, which involves the construction of advanced laboratories and production plants to prevent the dispersal of the virus; the need for purification of the viral antigens in order to distinguish between infected and vaccinated animals; the need for cold-chain transportation and low-temperature storage to ensure the stability of the antigens; and worse, the incomplete inactivation of the virus may lead to the risk of epidemics. Thus, there is an urgent need to develop a new universal or broad-spectrum vaccine for FMD.

FMD vaccine-induced immune protection is mainly mediated by neutralizing antibodies against viral structural proteins. However, it has been found that immunized animals can still resist infection in the absence or at very low potency of neutralizing antibodies, which shows that cellular immunity may play an important role in this regard. Recent studies have also confirmed that the more conserved nonstructural proteins 2B, 2C, 3A, 3B, 3C, and 3D proteins in different serotypes of FMDV can be recognized by bovine or porcine T-cells. T-cell immunity plays a crucial role in antiviral immunity. However, specific T cell antigenic epitopes have not been fully characterized.

Studies of the cellular immune response to FMDV have shown that FMDV infection activates the T-cell immune response in cattle and pigs. The degree of T-cell activation caused by infection is greater than that of vaccination. Proliferation of virus-specific CD4 T-cells is related to the increase in neutralizing antibody titers, and the virus-specific cytotoxic CD8 T-cells (CTLs) induced by FMDV wild-type strain infection in pigs can last for 5 weeks. The FMDV P12A-3C gene is expressed using a human adenoviral vector, and the animals immunized with recombinant virus can induce antigen-specific CTL response. Induction of epitope-specific CD8 T cells by vaccination with a vaccine containing a conserved antigenic epitope reduces the severity and duration of FMD disease. Using overlapping peptides and enzyme-linked immunospot (ELISPOT) assays, T cell epitopes of FMDV nonstructural proteins have been screened. Three conserved T-cell epitopes (aa 11-25, 21-35, 166-180) and one conserved T-cell epitope (aa 196-210) are respectively identified on the FMDV 3A protein and the 3C protein, while five T-cell epitopes are found in the 3D protein. These studies have shown that integrating FMDV T-cell epitopes into vaccines significantly improves the immunoprotective effect of vaccines. Thus, identifying the T-cell epitopes of non-structural proteins of FMDV and clarifying the phenotype and function of non-structural protein-specific T cells will help develop a broad-spectrum universal vaccine of targeted T cells against FMD and improve the cross-protection ability of existing vaccines against different FMDV serotypes.

### SUMMARY

The disclosure utilizes immunological techniques such as multi-color flow cytometry, enzyme-linked immunosorbent assay, intracellular cytokine staining, and in vitro culture of T cells to analyze the phenotype and function of specific porcine T-cells binding to FMDV nonstructural protein. In vitro culture of porcine CD4T and CD8 T cells specific for FMDV non-structural protein peptides provides immunological rationale for the development of a novel broad-spectrum vaccine against FMDV.

The disclosure screens the T-cell immune antigenic epitopes of two conserved non-structural proteins 2C and 2B of foot and mouth disease virus (FMDV), to obtain a T-cell epitope polypeptide of the non-structural proteins of FMDV, which has a high degree of sequence conservatism, and can efficiently induce an FMDV-specific immune response.

In one aspect, the first objective of the disclosure is to provide a T-cell epitope polypeptide of foot and mouth disease virus (FMDV), the epitope polypeptide having an amino acid sequence represented by one of SEQ ID Nos: 1-11:

SEQ ID NO: 1: KGKPFNSKVIIATTNLYS;
SEQ ID NO: 2: PLQNVYQLVQEVIDRVEL;
SEQ ID NO: 3: VVMDDLGQNPDGKDFKYF;
SEQ ID NO: 4: YNQQTVVVMDDLGQNPDG;
SEQ ID NO: 5: GRTDSVWYCPPDPDHFDG;
SEQ ID NO: 6: RGKSGQGKSFLANVLAQA;
SEQ ID NO: 7: PDFNRLVSAFEELATGVK;
SEQ ID NO: 8: AIRTGLDEAKPWYKLIKL;
SEQ ID NO: 9: MSTKHGPDFNRLVSAFEE;
SEQ ID NO: 10: MLDGRTMTDSDYRVF;
and
SEQ ID NO: 11: VLDEVIFSKHKGDTK.

In a class of this embodiment, the epitope polypeptide having an amino acid sequence represented by one of SEQ ID Nos: 3, 6, and 7.

In a class of this embodiment, the T-cell epitope polypeptide of foot and mouth disease virus comprises a 80-100% homologous sequence polypeptide.

In a second aspect, the disclosure provides a construct comprising the nucleic acid molecule encoding the T-cell epitope polypeptide of FMDV

In a third aspect, the disclosure provides a host cell comprising the construct and/or a cell transformed or transfected by the construct.

In a fourth aspect, the disclosure provides a composition comprising the T-cell epitope polypeptide of FMDV and a pharmaceutically acceptable vector or auxiliary.

In a class of this embodiment, the composition further comprises a recombinant protein comprising a T-cell epitope polypeptide of FMDV, RNA or DNA of an antigenic polypeptide of FMDV, or a vector for expression of the antigenic polypeptide by an attenuated virus or bacterium.

In a class of this embodiment, a fusion protein of the epitope polypeptide has a sequence homology of 80-100%.

In a fifth aspect, the disclosure provides a pharmaceutical composition comprising the T-cell epitope polypeptide of FMDV, the construct, the host cell, or the composition, and a pharmaceutically acceptable auxiliary.

In a sixth aspect, the disclosure provides an antibody comprising the T-cell epitope polypeptide of FMDV.

In a class of this embodiment, the antibody further comprises a nucleic acid molecule of an antigen binding fragment of the antibody.

In a seventh aspect, the disclosure provides an immune composition, comprising the T-cell epitope polypeptide of FMDV and an adjuvant.

In a class of this embodiment, the immune composition is in the form of a vaccine, a detection reagent, or a biological diagnostic reagent.

In a class of this embodiment, the vaccine is an amino acid vaccine or a nucleic acid vaccine.

The following advantages are associated with the disclosure. The antigen epitope peptides provided by the disclosure can stimulate strong cellular immune responses and have good immune effects, producing better neutralizing antibodies and complete protection.

### BRIEF DESCRIPTION OF THE DRAWINGS

FIG. 1 shows 15 amino acid short peptide from conserved regions of L, VP1, VP2, VP3, and overlapping peptides of 2B and 2C;

FIG. 2 shows clinical symptoms score of infected pigs;

FIG. 3 shows ELISPOT screening of single peptides of pigs after 14 days post-infection;

FIG. 4 shows ELISPOT screening of single peptides of pigs after 28 days post-infection;

FIG. 5 shows different T-cell epitopes recognized by different infected pigs;

FIG. 6 shows analysis of IFN-γ secretion and Ki67 proliferation in CD4⁺ T cells through intracellular cytokine staining;

FIG. 7 shows analysis of IFN-γ secretion and Ki67 proliferation in CD8⁺ T cells through intracellular cytokine staining; and

FIG. 8 shows epitope information that triggers activation of CD4 or CD8 T cells.

### DETAILED DESCRIPTION

To further illustrate the disclosure, embodiments detailing a T-cell epitope polypeptide of the foot and mouth disease virus are described below. It should be noted that the following embodiments are intended to describe and not to limit the disclosure.

The experimental methods in the following examples, unless otherwise stated, are conventional methods. The experimental materials used in the following examples, unless otherwise stated, are commercially available through conventional means.

### Example 1 T-Cell Epitope Screening

### 1. Design and Synthesis of Overlapping Short Peptides

137 short peptides with 18 amino acids in length, overlapping 6 amino acids, covering 2B, 2C, and some other conserved regional epitopes, were synthesized through Nanjing Kingsley Company Limited. Each peptide was dissolved to a concentration of 1 mg/mL. Each peptide was dissolved separately at a concentration of 0.1 mg/mL to yield 0.1 mg/mL of peptide pools or peptide mixtures, and the dissolved peptides were stored in a −80° C. refrigerator.

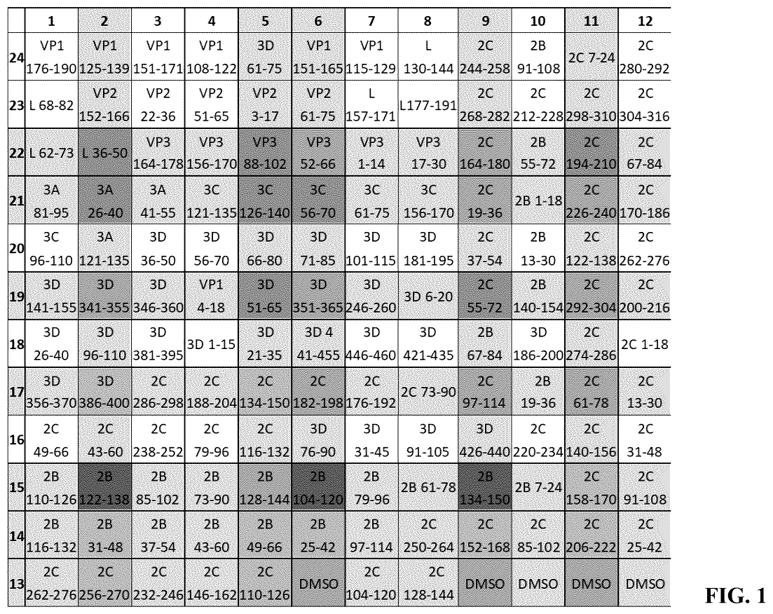

To screen T-cell epitopes from the nonstructural proteins 2B and 2C of FMDV, a total of 137 peptides of FMDV 2B and 2C were synthesized, and then 24 peptides were mixed according to the orthogonal matrix, and the design of the peptide library is shown in FIG. 1.

### 2. Animal Experiment Design

Fattening pigs of about 50 kg were obtained from Lanzhou Veterinary Research Institute, Chinese Academy of Agricultural Sciences (LVRI, CAAS). All animal care and feeding followed the ethical review system of LVRI, CAAS, and infection experiments were conducted in the animal high-level P3 laboratory. Three pigs were infected with 1000 ID₅₀ of foot-and-mouth disease (FMD) (O/BY/2010) virus, and one pig was injected with PBS as a control group. Clinical signs were observed at 3, 5, and 7 days after infection, and viremia was tested at 3, 7, and 10 days, followed by peripheral blood collection at 7, 14, and 28 days. The detection is carried out using ELISPOT assay.

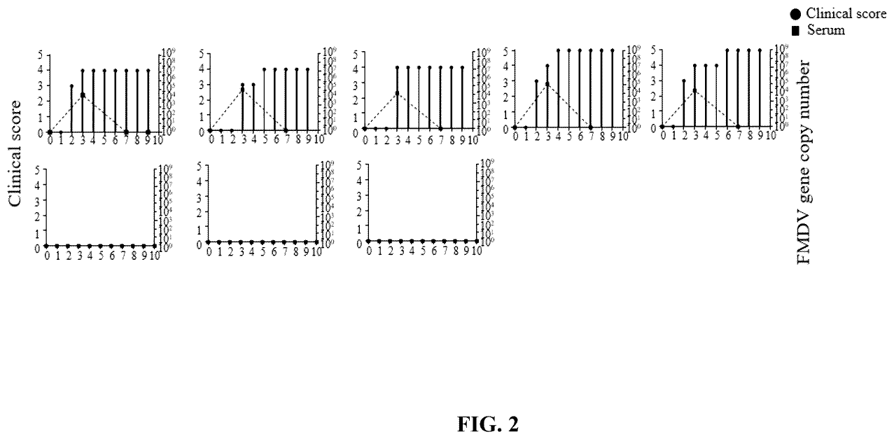

The pigs in the virus group had blister formation on the lips, tongues, gums, noses, or extremities and loss of appetite. Viremia was detected in all three pigs on the third day post infection and was undetectable on the seventh day. The control group was asymptomatic and had no viremia. Three sick pigs recovered on the tenth day, but still had loss of appetite. Five pigs were infected with O-FMDV and three were injected with PBS. The infection success of the pigs was assessed by recording clinical signs of infection (left side) and by detecting viremia in the blood (right side). Moreover, clinical signs could be observed 2-3 days after infection and peak viremia was detected on the third day, as shown in FIG. 2.

### 3. PBMC Separation

Porcine PBMC were isolated using the porcine peripheral blood mononuclear cell isolation kit (Solarbio) according to the manufacturer's instructions. Typically, peripheral porcine blood was obtained and diluted with two 4 mL of an anticoagulant (e.g., EDTAK2) through equal volumes of cell and tissue diluents. The diluted blood was then added to an equal volume of isolation medium (volume of blood and diluent) and centrifuged at 1000 g for 30 minutes at room temperature. After centrifugation, a milky white lymphocyte layer was observed between the dilution layer and the isolation medium layer. The lymphocyte layer was separated and washed twice with PBS. The washing step was performed by centrifugation at 300 g for 10 min at room temperature. PBMC of each pig was resuspended in a serum-free medium for ELISPOT assay (Mabtch) and calculated for use.

### Example 2 Identification of T-Cell Epitopes

### 1. Identification of T-Cell Epitopes Through ELISPOT

Porcine IFN-γ Elispotplus (HRP) Kit (Mabtech); reactions with pigs were carried out according to kit instructions; the plates pre-coated with anti-porcine IFN-g Ab were washed five times using 200 mL of sterile PBS and the coated plates were incubated with 10 mL of serum-free DMEM for at least 30 minutes at room temperature. After removal of the medium, stimuli containing different single peptides 2B and 2C together with porcine 5×10⁵ PBMC cells were added to each well, and the final concentrations of the stimuli were as follows: each peptide in the peptide pool or peptide mixture was 5 mg/mL, and individual peptide was 40 mg/mL. Finally, the cells were incubated for 36 hours at 37° C. in an incubator with 5% CO₂.

The plates were washed 5 times with sterile PBS, biotinylated detection antibody having a concentration of 0.5 g/mL was added, and incubated in an incubator comprising 5% CO₂ at 37° C. for 2 h. Subsequently, the plates were further washed 5 times with sterile PBS, and biotinylated HRP solution was added and incubated at room temperature for 1 h. Thereafter, the TMB substrate solution was added for color development for 2-20 min, and washed with a large amount of deionized water. The number of spots was read using an AID ELISpot machine and stored in a dark environment.

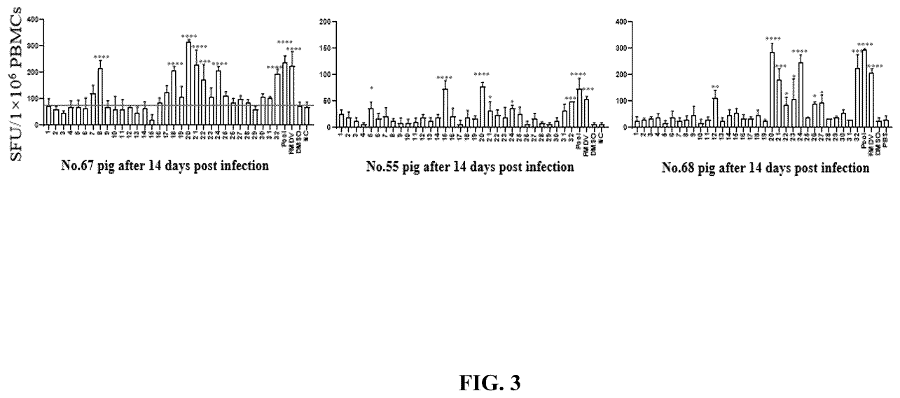

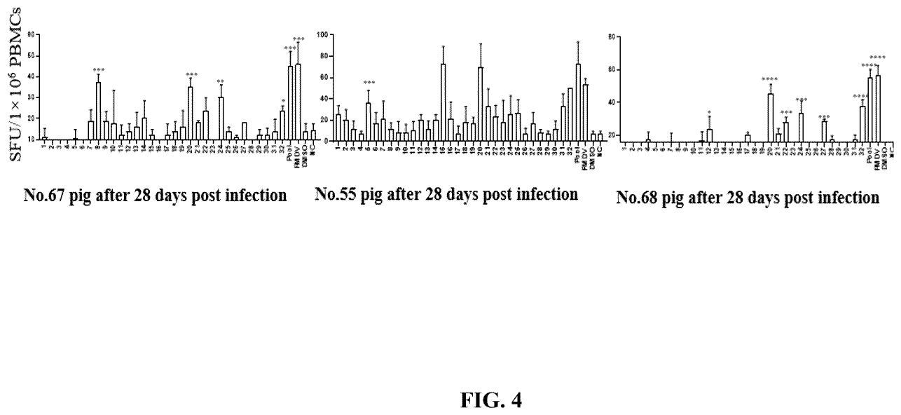

On the 14^{th} and 28^{th} days post infection, PBMC were isolated for ELISPOT test. The screened peptides induced the production of obvious immune spots, and the total number of the spots produced in each pig was counted separately (see FIGS. 3 and 4).

As shown in FIG. 3, a strong T cell response was detected by IFN-γ ELISPOT on the 14^{th} day after FMDV virus infection, with positive response peptides screened in 32 single peptide stimuli from a library of 24 peptides.

As shown in FIG. 4, a significant decrease in T cell responses was detected by IFN-γ ELISPOT on the 28^{th} day after FMDV virus infection relative to the 14^{th} day.

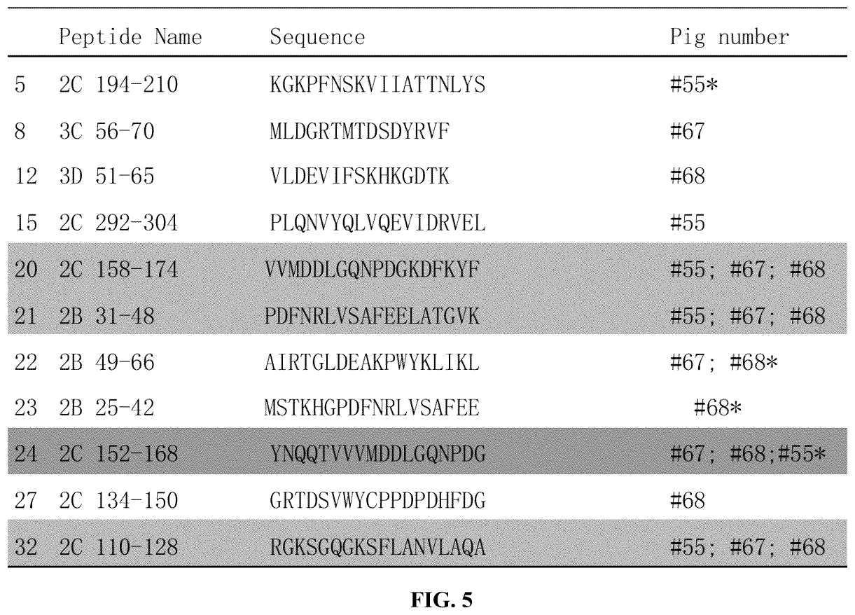

Epitopes recognized by T cells from different pigs were found in the non-structural proteins 2B, 2C, 3C, and 3D, as shown in FIG. 5, and their sequences are listed in Table 1.

TABLE 1
Amino acid sequence

| Non- | | |
|---|---|---|
| structural | Sequence | Amino acid |
| protein | name | sequence |
| 2C | SEQ ID NO. 1 | KGKPFNSKVIIATTNLYS |
| | SEQ ID NO. 2 | PLONVYQLVQEVIDRVEL |
| | SEQ ID NO. 3 | VVMDDLGQNPDGKDFKYF |
| | SEQ ID NO. 4 | YNQQTVVVMDDLGQNPDG |
| | SEQ ID NO. 5 | GRTDSVWYCPPDPDHFDG |
| | SEQ ID NO. 6 | RGKSGQGKSFLANVLAQA |
| | SEQ ID NO. 7 | PDFNRLVSAFEELATGVK |
| 2B | SEQ ID NO. 8 | AIRTGLDEAKPWYKLIKL |
| | SEQ ID NO. 9 | MSTKHGPDFNRLVSAFEE |
| 3C | SEQ ID NO. 10 | MLDGRTMTDSDYRVF |
| 3D | SEQ ID NO. 11 | VLDEVIFSKHKGDTK |

### 2. Flow Cytometry Validation Assay

At 14 days post-infection, anticoagulated blood was collected and porcine peripheral blood mononuclear cells were isolated, stained and counted by Taipan blue. 1×10⁶ cells were cultured in complete medium 1640 (RPMI 1640 comprising 10% FBS, 10 mM HEPES, 0.1 mM 3-ME, 0.1 mM non-essential amino acids, 0.1 mM sodium pyruvate, 2 mM L-glutamine and penicillin-streptomycin), and stimulated with 1 g/mL of peptide for 6 h, and 3 g/mL of BFA (brefeldin A, BFA) was added.

The cells were stained on the surface at room temperature for 10 min with a mixture of CD8α-BV421, CD4 FITC, CD3-PE-Cy7 and live/dead cell antibodies (Vitaly Dye Red 780, Thermo). After surface staining was completed, membrane-breaking and fixation were performed for 15 min at room temperature using BD's membrane-breaking staining solution. After fixation, staining was performed for 30 min at room temperature using IFNγ-APC and TNFα-PE antibodies in perm/wash buffer. Finally, data were collected using a BD LSRFortessa flow instrument and analyzed with the software Flowjo.

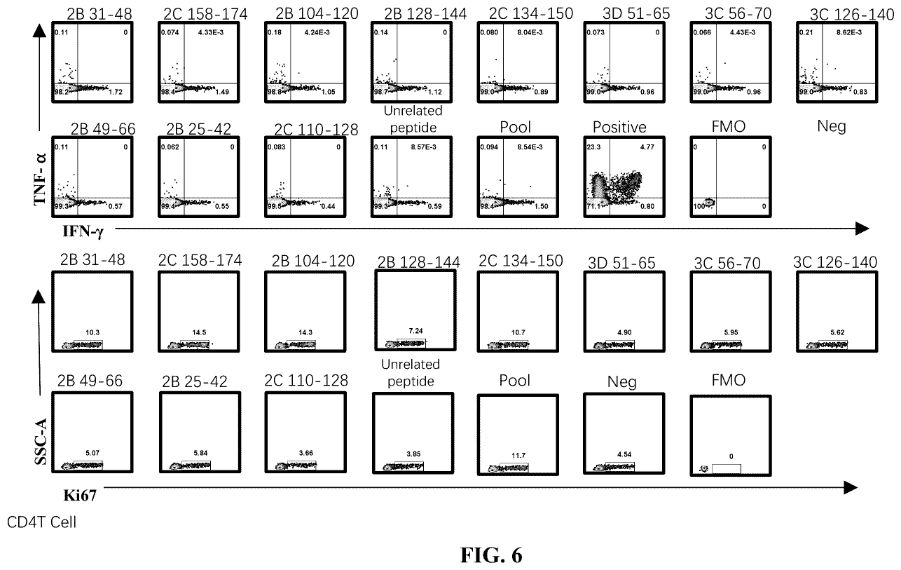

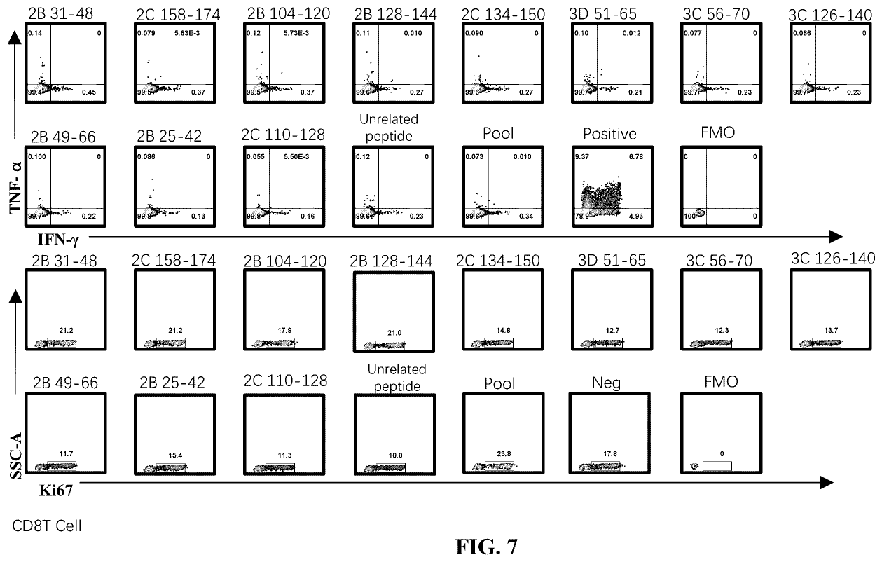

As shown in FIGS. 6 and 7, PBMC cells were isolated from pigs on the 14^{th} day post-infection, stained with intracellular, intranuclear, and extracellular markers and analyzed by FACS, and the PBMC cells from infected pigs were stimulated with peptides screened by ELISpot. MHC-II restriction of the identified peptides was determined by intracellular factor staining (IFN-7) and intranuclear staining (Ki67).

Standard ICS analyses were performed to investigate the phenotype of specific T cells promoting the secretion of IFN-7. The results showed that all six strong positive response peptides identified in the ELISPOT assay induced a significant increase in the level of IFN-γ secretion in CD4⁺ T cells compared to the negative control, suggesting that they contain potential CD4⁺ T cell epitopes.

### 3. Ki67 Cell Proliferation Assay

Single cell suspension was prepared as described above, and stimulated for 72 h using 1 g/mL of the peptide for staining analysis. Specifically, the cells were stained with Thermo's LIVE/DEAD Violet for 30 min to differentiate between live and dead cells; the cells were subsequently stained in 1% FCS/PBS buffer containing BD's anti-pig CD3, CD4, CD8α, γδ T antibodies for 15 min, washed for 15 min, and fixed in BD's Cell Membrane/Cell Treatment Solution for 15 min. Finally, intranuclear staining was performed in PERM/WASH buffer using BD's Ki67 antibody for 20 min at 4° C., and data were collected by a BD LSRFortessa flow instrument and analyzed by the software Flowjo.

As shown in FIGS. 6 and 7, after the CD4+ and CD8⁺ T cells were stimulated with T cell epitope peptides at 41° C. and 5% CO₂ for 3 days, a Ki67 staining assay was performed to evaluate the proliferation of CD4+ and CD8⁺ T cells. The results showed that the six-strong positive response peptides identified in the ELISPOT assay induced a significant increase in the proliferation rate of CD4⁺ T cells in FMDV-infected porcine PBMC. Consistent with the data from ICS analysis, CD4⁺ T cell proliferation was also induced.

### 4. Conservative Analysis of T-Cell Epitopes

The genome sequences of 46 FMDV nonstructural proteins 2B and 2C were downloaded from GenBank, and multi-amino acid sequence alignment was performed using Genetic Prime. Comparative analysis was performed on the obtained T cell epitopes, and finally, using Geneious Prime software, a region spanning the identified epitope sequence was used to generate the logo.

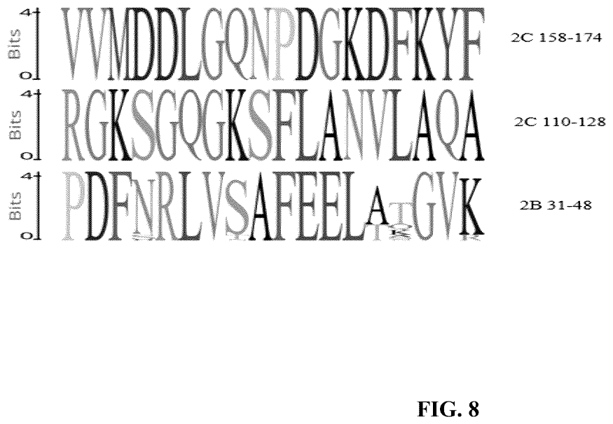

To analyze the conservation and broad spectrum of the screened FMDV 2C and 2B T-cell epitopes, the 7 FMDV amino acid sequences currently available in the NCBI database were downloaded and aligned using Geneious Prime software to determine the number of virulent strains recognized by the epitopes as well as the conservation analysis (shown in FIG. 8), and the sequence list is shown in Table 2.

TABLE 2
Amino acid sequence

| Non-structural | Sequence | Amino acid |
|---|---|---|
| protein | name | sequence |
| | SEQ ID NO. 3 | VVMDDLGQNPDGKDFKYF |
| 2C | SEQ ID NO. 6 | RGKSGQGKSFLANVLAQA |
| 2B | SEQ ID NO. 7 | PDFNRLVSAFEELATGVK |

### Example 3 Construction and Immune Efficacy Evaluation of FMDV T/B Vaccine

The experimental pigs were divided into four groups, one group immunized with FMD T/B epitope, one group immunized with B-cell epitope vaccine, one group immunized with T epitope vaccine, and one group not immunized as a negative control. The initial and booster immunization strategies were used, and the virus was attacked 7 days after the booster immunization to observe the onset of the disease and detect the viral loads of blood, oral cavity, nasal swabs, intestinal swabs, and important immune organs of the pigs. Differences in indicators between groups were compared to determine the protective effect of T-cell epitope nanoparticle vaccines.

The screened peptide was fused with a B-cell epitope (VP1 GH-loop), expressed and purified in E. coli. Through the guinea pig immunization experiments, the result demonstrated that the B-lymphoid epitope, fused with T-lymphocyte epitopes 20, 21, 24, and 32, induced the body to produce higher levels of neutralizing antibodies (see Table 3).

TABLE 3
Neutralization Effects of TB Vaccines

| Experimental group | No. | 14 day | 21 day | 28 day |
|---|---|---|---|---|
| 201 + FMDV TB | 1 | 1:8 | 1:256 | 1:1024 |
| 201 + FMDVT | 2 | 1:8 | 1:256 | 1:1024 |
| | 3 | 1:8 | 1:256 | 1:1024 |
| | 4 | 1:8 | 1:128 | 1:512 |
| | 5 | 1:8 | 1:256 | 1:1024 |
| | 6 | 1:8 | 1:512 | 1:1024 |
| | 7 | <1:4 | <1:4 | <1:4 |
| | 8 | <1:4 | <1:4 | <1:4 |
| | 9 | <1:4 | <1:4 | <1:4 |
| | 10 | <1:4 | <1:4 | <1:4 |
| | 11 | <1:4 | <1:4 | <1:4 |
| 201 + FMDV B | 12 | <1:4 | 1:16 | 1:32 |
| | 13 | <1:4 | 1:16 | 1:64 |
| | 14 | <1:4 | 1:8 | 1:64 |
| | 15 | <1:4 | 1:16 | 1:64 |
| | 16 | <1:4 | 1:16 | 1:32 |
| Ctrl | 17 | <1:4 | <1:4 | <1:4 |
| | 18 | <1:4 | <1:4 | <1:4 |
| | 19 | <1:4 | <1:4 | <1:4 |

It will be obvious to those skilled in the art that changes and modifications may be made, and therefore, the aim in the appended claims is to cover all such changes and modifications.

What is claimed is:

1. A T-cell epitope polypeptide of foot and mouth disease virus (FMDV), the epitope polypeptide having an amino acid sequence represented by one of SEQ ID Nos: 1-11:
SEQ ID NO: 1: KGKPFNSKVIIATTNLYS;
SEQ ID NO: 2: PLQNVYQLVQEVIDRVEL;
SEQ ID NO: 3: VVMDDLGQNPDGKDFKYF;
SEQ ID NO: 4: YNQQTVVVMDDLGQNPDG;
SEQ ID NO: 5: GRTDSVWYCPPDPDHFDG;
SEQ ID NO: 6: RGKSGQGKSFLANVLAQA;
SEQ ID NO: 7: PDFNRLVSAFEELATGVK;
SEQ ID NO: 8: AIRTGLDEAKPWYKLIKL;
SEQ ID NO: 9: MSTKHGPDFNRLVSAFEE;
SEQ ID NO: 10: MLDGRTMTDSDYRVF;
and
SEQ ID NO: 11: VLDEVIFSKHKGDTK.

2. The epitope polypeptide of claim 1 having an amino acid sequence represented by one of SEQ ID Nos: 3, 6 and 7.

3. A construct, comprising a nucleic acid molecule encoding the T-cell epitope polypeptide of FMDV of claim 1.

4. A host cell, comprising the construct of claim 3, and/or a cell transformed or transfected by the construct.

5. A composition, comprising the T-cell epitope polypeptide of FMDV of claim 1 and a pharmaceutically acceptable vector or auxiliary.

6. The composition of claim 5, further comprising a recombinant protein comprising a T-cell epitope polypeptide of FMDV, RNA or DNA of an antigenic polypeptide of FMDV, or a vector for expression of the antigenic polypeptide by an attenuated virus or bacterium.

7. A pharmaceutical composition comprising the T-cell epitope polypeptide of FMDV of claim 1, a construct comprising the T-cell epitope polypeptide of FMDV, a host cell comprising the construct, or a composition comprising the T-cell epitope polypeptide of FMDV, and a pharmaceutically acceptable auxiliary.

8. An antibody comprising the T-cell epitope polypeptide of FMDV of claim 1 and a nucleic acid molecule of an antigen-binding fragment of the antibody or a vector or host cell comprising the nucleic acid molecule.

9. An immune composition comprising the T-cell epitope polypeptide of FMDV of claim 1 and an adjuvant.

10. The immune composition of claim 9, being in the form of a vaccine, a detection reagent, or a biological diagnostic reagent.


SEQUENCE LISTING (11 sequences)

SEQ ID NO: 1 - protein, 18 aa, synthetic construct
KGKPFNSKVIIATTNLYS

SEQ ID NO: 2 - protein, 18 aa, synthetic construct
PLQNVYQLVQEVIDRVEL

SEQ ID NO: 3 - protein, 18 aa, synthetic construct
VVMDDLGQNPDGKDFKYF

SEQ ID NO: 4 - protein, 18 aa, synthetic construct
YNQQTVVVMDDLGQNPDG

SEQ ID NO: 5 - protein, 18 aa, synthetic construct
GRTDSVWYCPPDPDHFDG

SEQ ID NO: 6 - protein, 18 aa, synthetic construct
RGKSGQGKSFLANVLAQA

SEQ ID NO: 7 - protein, 18 aa, synthetic construct
PDFNRLVSAFEELATGVK

SEQ ID NO: 8 - protein, 18 aa, synthetic construct
AIRTGLDEAKPWYKLIKL

SEQ ID NO: 9 - protein, 18 aa, synthetic construct
MSTKHGPDFNRLVSAFEE

SEQ ID NO: 10 - protein, 15 aa, synthetic construct
MLDGRTMTDSDYRVF

SEQ ID NO: 11 - protein, 15 aa, synthetic construct
VLDEVIFSKHKGDTK